(19) United States
(12) Patent Application Publication
(10) Pub. No.: US 2017/0348361 A1
(43) Pub. Date: Dec. 7, 2017

(54) PROBIOTIC AUGMENTATION OF ANTI-TUMOR ENDOTHELIUM IMMUNE RESPONSES

(71) Applicant: Batu Biologics, Inc., San Diego, CA (US)

(72) Inventors: Samuel C. Wagner, San Diego, CA (US); Thomas E. Ichim, San Diego, CA (US)

(73) Assignee: Batu Biologics, Inc., San Diego, CA (US)

(21) Appl. No.: 15/611,710

(22) Filed: Jun. 1, 2017

Related U.S. Application Data
(60) Provisional application No. 62/344,887, filed on Jun. 2, 2016.

(51) Int. Cl. A61K 35/741; A61K 9/107; A61K 35/747; A61K 35/744; A61K 35/74; A61K 35/745; A61K 9/06; A61K 35/00; A61K 45/00

(52) U.S. Cl. CPC A61K 35/741; A61K 35/745; A61K 9/107; A61K 35/747; A61K 9/06; A61K 35/744; A61K 35/74; A61K 45/00; A61K 2035/115

(57) ABSTRACT

Augmenting or stimulating an immune response, to tumor endothelial cells, by: a) obtaining a tumor endothelial antigen or composition of antigens; b) administering said tumor endothelial antigen or composition of antigens in an immunogenic manner to a host; and c) providing a probiotic and/or prebiotic mixture to said host being immunized.

### CROSS-REFERENCE TO RELATED APPLICATION

This application claims the benefit of U.S. Provisional Application No. 62/344,887 filed on Jun. 2, 2016, entitled “PROBIOTIC AUGMENTATION OF ANTI-TUMOR ENDOTHELIUM IMMUNE RESPONSES”, the contents of which are incorporated herein by reference as though set forth in their entirety.

### FIELD OF THE INVENTION

The invention pertains to field of probiotics. More particularly the invention pertains to the use of probiotics and prebiotics to augment immune responses towards tumor endothelial cells.

### BACKGROUND

The immune system is comprised of multiple different cell types, biologically active compounds and molecules and organs. These include lymphocytes, monocytes and polymorphonuclear leukocytes, numerous soluble chemical mediators (cytokines and growth factors), the thymus, postnatal bone marrow, lymph nodes, liver and spleen. All of these components work together through a complex communication system to fight against microbial invaders such as bacteria, viruses, fungi and parasites, and tumor cells. Together, these components recognize specific molecular antigens as foreign or otherwise threatening, and initiate an immune response against cells or viruses that contain the foreign antigen. The immune system also functions to eliminate damaged or cancerous cells through active surveillance using the same mechanisms used to recognize microbial or viral invaders. The immune system recognizes the damaged or cancerous cells via antigens that are not strictly foreign, but are aberrantly expressed or mutated in the targeted cells.

### SUMMARY OF THE INVENTION

A method of augmenting an immune response to tumor endothelial cells, the method may comprise the steps: a) obtaining a tumor endothelial antigen or composition of antigens; b) administering the tumor endothelial antigen or composition of antigens in an immunogenic manner to a host; and c) providing a probiotic and/or prebiotic mixture to the host being immunized. The antigen expressed on tumor endothelial cells may be selected from a group comprising: a) ROBO-4; b) VEGF-R2; c) FGF-R; d) CD105; e) TEM-1; f) survivin; g) CD93; h) CD 109; and i) ROBO 1-18. The antigenic composition may comprise of ValloVax. The probiotic administered may be Lactobacillus kefiranofaciens. The probiotic administered may be a strain of Lactobacillus kefiranofaciens selected from the group consisting of R2C2, INIX, K2, BioSP and ES1. The probiotic administered may be Bifidobacteria. The Bifidobacteria may be Bifidobacterium NCIMB 41676. The probiotic administered may be Lactobacillus salivarius. The prebiotic administered may be fructose polymers GFₙ and Fₘ, either containing a glucose (G) end-group, or without a glucose end-group, and one or more component of a group of prebiotics consisting of modified or unmodified starch and partial hydrolysates thereof, partially hydrolysed inulin, natural oligofructoses, fructo-oligosaccharides (FOS), lactulose, galactomannan and suitable partial hydrolysates thereof, indigestible polydextrose, acemannan, various gums, indigestible dextrin and partial hydrolysates thereof, trans-galacto-oligosaccharides (GOS), xylo-oligosaccharides (XOS), beta-glucan and partial hydrolysates thereof, together if desired with phytosterol/phytostanol components and their suitable esters, and if desired other plant extracts, mineral components, vitamins and additives. The probiotic administered may be selected from a group comprising: a) Lactobacillus; b) Leuconostoc; c) Pediococcus; d) Lactococcus,; e)Aerococcus; f) Carnobactehum; g) Enterococcus; h) Oenococcus; i) Teragenococcus; j) Vagococcus, and h) Weisella. The probiotic bacteria may be selected from a group, alone or in combination, comprising: a) Streptococcus thermophiles; b) Lactobacillus reuteri; c) Bifidobacterium bifidium; d) Latobacillus acidophilus; and e) Latobacillus casei.

### DETAILED DESCRIPTION OF THE INVENTION

In one embodiment, specific probiotic bacteria are administered individually or in combination with tumor endothelial antigens, or polyvalent mixtures containing tumor endothelial antigens. In one embodiment, lactic acid bacterium and/or Bifidobacterium are administered at a concentration of 10(6) to 10(12) colony forming units (CFU) of bacteria per gram of support material, and more particularly from 10(8) to 10(12) CFU of bacteria/gram of support material, preferably 10(9) to 10(12) CFU/gram of support material for the lyophilized form. In the specific embodiment, said bacterium is administered orally, at a frequency sufficient to induce immune modulation. Specifically, immune modulation comprises upregulation of Th1 cytokines, and ability of vaccination with tumor endothelial specific antigens or composition of antigens. In one embodiment, ValloVax is administered subsequent to initiation of probiotic treatment. In one embodiment, administration of probiotics is performed daily for two weeks prior to ValloVax immunization.

Suitably the lactic acid bacterium and/or Bifidobacterium used in accordance with the present invention may be administered at a dosage of from about 10⁶ to about 10¹² CFU of microorganism/dose, preferably about 10⁸ to about 10¹² CFU of microorganism/dose. By the term “per dose” it is meant that this amount of microorganism is provided to a subject either per day or per intake, preferably per day. For example, if the microorganism is to be administered in a food product (for example in a yoghurt)—then the yoghurt will preferably contain from about 10⁸ to 10¹² CFU of the microorganism. Alternatively, however, this amount of microorganism may be split into multiple administrations each consisting of a smaller amount of microbial loading—so long as the overall amount of microorganism received by the subject in any specific time (for instance each 24 hour period) is from about 10⁶ to about 10¹² CFU of microorganism, preferably 10⁸ to about 10¹² CFU of microorganism.

In accordance with the present invention, an effective amount of at least one strain of a microorganism may be at least 10⁶ CFU of microorganism/dose, preferably from about 10⁶ to about 10¹² CFU of microorganism/dose, preferably about 10⁸ to about 10¹² CFU of microorganism/dose. In one embodiment, preferably the lactic acid bacterium and/or Bifidobacterium used in accordance with the present invention (such as a strain of Lactobacillus spp.; for example a strain of Lactobacillus acidophilus, Lactobacillus salivarius and/or Lactobacillus plantarum and/or a strain of Bifidobacterium spp., such as a strain of Lactobacillus acidophilus or Lactobacillus salivarius, for example Lactobacillus acidophilus strain such as NCFM or Lactobacillus salivarius strain 33) such as a strain of Bifidobacterium animalis subsp. lactis, for example Bifidobacterium animalis subsp. lactis strain 420 (B420)) may be administered at a dosage of from about 10⁶ to about 10¹² CFU of microorganism/day, preferably about 10⁸ to about 10¹² CFU of microorganism/day. Hence, the effective amount in this embodiment may be from about 10⁶ to about 10¹² CFU of microorganism/day, preferably about 10⁸ to about 10¹² CFU of microorganism/day.

The probiotic mixtures may be used according to the present invention in the form of solid or liquid preparations or alternatives thereof. Examples of solid preparations include, but are not limited to tablets, capsules, dusts, granules and powders which may be wettable, spray-dried or freeze-dried. Examples of liquid preparations include, but are not limited to, aqueous, organic or aqueous-organic solutions, suspensions and emulsions. Suitable examples of forms include one or more of: tablets, pills, capsules, ovules, solutions or suspensions, which may contain flavouring or colouring agents, for immediate-, delayed-, modified-, sustained-, pulsed- or controlled-release applications. By way of example, if the composition of the present invention is used in a tablet form—such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycoliate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included. Furthermore, examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethylcellulose, polyvinylpyrrolidone, and the like. Preferred excipients for the forms include lactose, starch, a cellulose, milk sugar or high molecular weight polyethylene glycols.

In one embodiment, probiotic bacteria are administered in the form of a nutraceutical. Nutraceuticals, whether in the form of a liquid extract or dry composition, are edible and may be eaten directly by humans or mammals. Said nutraceuticals are preferably provided to humans in the form of additives or nutritional supplements e.g., in the form of tablets of the kind sold in health food stores, or as ingredients in edible solids, more preferably processed food products such as cereals, breads, tofu, cookies, ice cream, cakes, potato chips, pretzels, cheese, etc., and in drinkable liquids e.g., beverages such as milk, soda, sports drinks, and fruit juices. Thus, in one embodiment a method is provided for enhancing the nutritional value of a food or beverage by intermixing the food or beverage with a nutraceutical in an amount that is effective to enhance the nutritional and probiotic or immune modulatory value of the food or beverage. In one embodiment, a flavoring agent is added. Preferred flavoring agents include sweeteners such as sugar, corn syrup, fructose, dextrose, maltodextrose, cyclamates, saccharin, phenyl-alanine, xylitol, sorbitol, maltitol, and herbal sweeteners such as Stevia. Examples of foods into which probiotics useful for the practice of the invention can be incorporated into include soft drinks, a fruit juice or a beverage comprising whey protein, health teas, cocoa drinks, milk drinks and lactic acid bacteria drinks. Probiotic bacteria may be administered together with agents known to enhance efficacy and retention of probiotics, including [0035] In a further embodiment of the present invention various extracts and plant powders are incorporated into our compositions, depending on the desired properties according to the end use of said compositions. These compositions according to the present invention can be characterized in that in addition to the discussed prebiotics and phytosterols and lecithins the said further plant extracts or powders are one or more of those of Panax ginseng (red, Korean ginseng), Panax ginseng (white, Chinese ginseng), Rhodiola rosea (golden root), Panax quinquefolium (American ginseng), Eleutherococcus senticosus (Siberian ginseng), Cynara scolymus (artichoke), Uncaria tomentosa (Cat's claw), Lepidium meyenii (maca, Peruvian ginseng), Paullinia cupana (guarana), Croton lechleri (Sangre de Grado), Whitania somnifera (ashwagandha, Indian ginseng), Panax japonicus (Japanese ginseng), Panax vietnamensis (Vietnamese ginseng), Panax trifolius, Panax pseudoginseng, Panax notoginseng, Malpighia glabra (acerola), Ylex paraguayiensis (Yerba mate), Astragalus membranaceus (astragalus), Stevia rebaudiana (stevia), Pfaffia paniculata (Brazilian ginseng, suma), Ginkgo biloba, Tabebuia impetiginosa (Pau d'arco), Echinacea purpurea, Peumus boldus (boldo), Gynostemma pentaphyllum (Jiaogulan, also known as Southern Ginseng or Xiancao), Sutherlandia frutescens (African ginseng), Aloe vera (aloe), Cistanche salsa, Cistanche deserticola (and other Cistanche sp.), Codonopsis pilosula (“poor man's ginseng.”), Nopal opuntia (Prickly pear cactus), Citrus sinensis (Citrus aurantium) and other members of the citrus family (lemon, lime, tangerine, grapefruit), Camelia sinensis (tea), Plantago psyllium (psyllium), Amaranth edulis and other amaranth sp. (amaranth), Commiphora mukul (guggul lipid), Serenoa repens, Serenoa serrulata (saw palmetto), Cordyceps sinensis (Cordycaps), Lentinula edodes (shitake), Ganoderma lucidium (Reishi), Grifola frondosa (maitake), Tremella fuciformis (Silver ear), Poria cocos (Hoelen), Hericium erinaceus (Lion's Mane), Agaricus blazei (Sun mushroom), Phellinus linteus (Mulberry yellow polypore), Trametes versicolo , Coriolus versicolor (Turkey tails), Schizophyllum commune (Split gill), Inonotus obliquus (Cinder conl), oat bran, rice bran, linseed, garlic, Ceratonia siliqua (locust been gum or flour from the seeds of carob tree), Cyanopsis tetragonoloba (guar gum, EU Food additive code E412), Xanthomonas campestris (xanthan gum). These plant extracts and plant powders are capable to potentiate the bioactivity of these compositions based on prebiotics, phytosterols, lecithins, vitamins and minerals. In given cases it also adds other prebiotics to the aforementioned prebiotic mixtures. These can result in more pronounced bioactivities as prebiotics and also in the chosen other bioactivity directions.

The nutraceuticals described herein are intended for human consumption and thus the processes for obtaining them are preferably conducted in accordance with Good Manufacturing Practices (GMP) and any applicable government regulations governing such processes. Especially preferred processes utilize only naturally derived solvents. In contrast to nutraceuticals, the so-called “medical foods” are not meant to be used by the general public and are not available in stores or supermarkets. Medical foods are not those foods included within a healthy diet to decrease the risk of disease, such as reduced-fat foods or low-sodium foods, nor are they weight loss products. A physician prescribes a medical food when a patient has special nutrient needs in order to manage a disease or health condition, and the patient is under the physician's ongoing care. The label must clearly state that the product is intended to be used to manage a specific medical disorder or condition. An example of a medical food is nutritionally diverse medical food designed to provide targeted nutritional support for patients with chronic inflammatory conditions. Active compounds of this product are for instance one or more of the compounds described herein. The present invention thus relates to the use of an immuno-modulating properties of probiotics as related to prevention and/treatment of pregnancy complications. Thus said probiotics can be used in the preparation of a medicament, a vaginal suppository, medical food or nutraceutical to induce immune tolerance or immune modulation.

In some embodiments, the compositions according to the present invention comprise prebiotic components selected from fructose polymers GFₙ and Fₘ, either containing a glucose (G) end-group, or without this glucose end-group and one or more component of a group of prebiotics consisting of modified or unmodified starch and partial hydrolysates thereof, partially hydrolysed inulin, natural oligofructoses, fructo-oligosaccharides (FOS), lactulose, galactomannan and suitable partial hydrolysates thereof, indigestible polydextrose, acemannan, various gums, indigestible dextrin and partial hydrolysates thereof, trans-galacto-oligosaccharides (GOS), xylo-oligosaccharides (XOS), beta-glucan and partial hydrolysates thereof, together if desired with phytosterol/phytostanol components and their suitable esters, and if desired other plant extracts, mineral components, vitamins and additives. The fructose polymers of GFₙ or Fₘ structures (G=glucose; F=fructose; n>2; m>2) are linear fructose polymers having either a glucose (G) and −group, or being without this glucose and -group. Oligofructoses are consisted of 3 to 10 carbohydrate units. Above that, chicory inulin contains 10 to 60 carbohydrate units, typically with 27 carbohydrates (fructoses with our without one glucose end-group and a fructose chain). Other plants may produce different fructans. These fructans are capable to increase the number of colonized and planktonic bacteria in the large intestine. This results in a change that those bacteria that are less advantageous or may turn dangerous are suppressed by the higher probiotic colony of bacteria. Depending on the chain length of these fructans or other prebiotics, they can be fermented by probiotic bacteria at different positions in the colon. We have found that the longer inulins are capable to rich the distal colon and sigmoid colon and exert their anticancer actions in the positions where typically most of the cancerous problems occur. The occurrence of these cancers can be the result of various types of carcinogenesis. It has been demonstrated in the literature that directly induced chemical carcinogenesis can be greatly reduced by probiotic bacteria. The prebiotic compositions of our invention can corroborate this effect by considerably increasing the number of Bifidocateria and other beneficial probiotic strains. The local chemical carcinogenesis can also be the result of the formation of secondary bile acids. These secondary bile acids are often formed upon the action of enzymes produced by resident Clostridia. By probiotic suppression of the number of these bacteria according to the invention, the chance of secondary bile acid formation can also be reduced. This can be demonstrated by measuring the faecal primary/secondary bile acid ratio. Other prebiotics can be selected from a group of prebiotics consisting of various gums (guar gum, xanthan gum, locust been gum), carob seed flour, oat bran, rice bran, barley, modified or unmodified starch and suitable partial hydrolysates thereof, partially hydrolysed inulin, natural or synthetic/biosynthetic oligofructoses, fructo-oligosaccharides (FOS), lactulose, galactomannan and suitable hydrolysates thereof, indigestible polydextrose, indigestible dextrin and partial hydrolysates thereof, trans-galacto-oligosaccharides (GOS), xylo-oligosaccharides (XOS), acemannan, lentinan or beta-glucan and partial hydrolysates thereof, polysaccharides P and K (PSP, PSK), tagatose, various fungal oligosaccharides and polysaccharides, together with other components.

Before or during the tumor endothelium vaccination protocol, the subjects are subjected to radiation directed at the tumor or, in some cases, to whole body irradiation. The effect of this radiation treatment is to induce remodeling of the vasculature so that extravasation of effector T cells into the tumor is enhanced. If the tumor to be treated is not a solid tumor or a tumor with defined lesions, this aspect of the protocol is optional and generally unnecessary. However, when radiation is utilized, probiotics are utilized to repair immune responses before vaccine administration. Additionally, prebiotics may be administered prior to irradiation.

The effect of radiation is to ease the entry of the effector T cells elicited by the vaccine into solid tumors, so that the radiation can be administered immediately before or during the vaccination protocol. The level of radiation dosage will depend on whether the tumor is targeted directly or whole body radiation is employed and on the level of remodeling that needs to be effected. The radiation schedule can be integrated with the schedule for administration of the vaccine and with the schedule for the administration of anti-CTLA-4 antibody that modulates the effect of Tregs. Each of the radiation treatments may be scheduled at a time selected to correspond to a particular administration of the vaccine and/or the Tregs modulator. In aspects of the invention, immunostimulatory oligonucleotides are administered together with probiotics. The immunostimulatory oligonucleotides can encompass various chemical modifications and substitutions, in comparison to natural RNA and DNA, involving a phosphodiester internucleoside bridge, a .beta.-D-ribose unit and/or a natural nucleoside base (adenine, guanine, cytosine, thymine, uracil). Examples of chemical modifications are known to the skilled person and are described, for example in Uhlmann E. et al. (1990), Chem. Rev. 90:543; “Protocols for Oligonucleotides and Analogs” Synthesis and Properties & Synthesis and Analytical Techniques, S. Agrawal, Ed., Humana Press, Totowa, USA 1993; Crooke, S. T. et al. (1996) Annu. Rev. Pharmacol. Toxicol. 36:107-129; and Hunziker J. et al., (1995), Mod. Synth. Methods 7:331-417.

1. A method of augmenting an immune response to tumor endothelial cells, said method comprising the steps: a) obtaining a tumor endothelial antigen or composition of antigens; b) administering said tumor endothelial antigen or composition of antigens in an immunogenic manner to a host; and c) providing a probiotic and/or prebiotic mixture to said host being immunized.

2. The method of claim 1, wherein said antigen expressed on tumor endothelial cells is selected from a group comprising:
a) ROBO-4;
b) VEGF-R2;
c) FGF-R;
d) CD105;
e) TEM-1;
f) survivin;
g) CD93;
h) CD 109; and
i) ROBO 1-18.

3. The method of claim 1, wherein said antigenic composition comprises of ValloVax.

4. The method of claim 1, wherein said probiotic administered is Lactobacillus kefiranofaciens.

5. The method of claim 4, wherein said probiotic administered is a strain of Lactobacillus kefiranofaciens selected from the group consisting of R2C2, INIX, K2, BioSP and ES1.

6. The method of claim 1, wherein said probiotic administered is Bifidobacteria.

7. The method of claim 6, wherein said Bifidobacteria is Bifidobacterium NCIMB 41676.

8. The method of claim 1, wherein said probiotic administered is Lactobacillus salivarius.

9. The method of claim 1, wherein said prebiotic administered is fructose polymers GFₙ and Fₘ, either containing a glucose (G) end-group, or without a glucose end-group, and one or more component of a group of prebiotics consisting of modified or unmodified starch and partial hydrolysates thereof, partially hydrolysed inulin, natural oligofructoses, fructo-oligosaccharides (FOS), lactulose, galactomannan and suitable partial hydrolysates thereof, indigestible polydextrose, acemannan, various gums, indigestible dextrin and partial hydrolysates thereof, trans-galacto-oligosaccharides (GOS), xylo-oligosaccharides (XOS), beta-glucan and partial hydrolysates thereof, together if desired with phytosterol/phytostanol components and their suitable esters, and if desired other plant extracts, mineral components, vitamins and additives.

10. The method of claim 1, wherein said probiotic administered is selected from a group comprising:
a) Lactobacillus;
b) Leuconostoc;
c) Pediococcus;
d) Lactococcus,;
e) Aerococcus;
f) Carnobactehum;
g) Enterococcus;
h) Oenococcus;
i) Teragenococcus;
j) Vagococcus, and
h) Weisella.

11. The method of claim 1, wherein said probiotic bacteria is selected from a group, alone or in combination, comprising:
a) Streptococcus thermophiles;
b) Lactobacillus reuteri;
c) Bifidobacterium bifidium;
d) Latobacillus acidophilus; and
e) Latobacillus casei.
